(19) United States
(12) Patent Application Publication
(10) Pub. No.: US 2012/0296196 A1
(43) Pub. Date: Nov. 22, 2012

(54) Method for assisting a person performing a minimally invasive intervention with a catheter involving a puncture of a septum and x-ray device

(76) Inventors: Jan BOESE, Eckental (DE); Matthias JOHN, Nurnberg (DE); Alois NÖTTLING, Pottenstein (DE); Thomas REDEL, Poxdorf (DE)

(21) Appl. No.: 13/474,797

(22) Filed: May 18, 2012

(30) Foreign Application Priority Data
May 20, 2011 (DE) 102011076217.5

(51) Int. Cl. A61B 6/00; G01N 23/223; A61B 5/055

(52) U.S. Cl. 600/411; 600/427; 378/44

(57) ABSTRACT

A method for assisting a person performing a minimally invasive intervention with a catheter involving a puncture of a septum, in particular of a heart, is proposed. A three-dimensional image data record is recorded showing an anatomical structure in the region of the septum. An item of septum information showing the position of the septum is determined in the image data record and additional information is derived from the position of the anatomical structure influencing or showing the selection of a puncture site. During the intervention, the fluoroscopic images of the region are continuously recorded and a current fluoroscopic image is displayed and superimposed with the septum information and/or the additional information based on a registration of the image data record with the fluoroscopic images.

### CROSS REFERENCE TO RELATED APPLICATIONS

This application claims priority of German application No. 10 2011 076 217.5 filed May 20, 2011, which is incorporated by reference herein in its entirety.

### FIELD OF INVENTION

The invention relates to a method for assisting a person performing a minimally invasive intervention with a catheter involving a puncture of a septum, in particular of a heart, and an X-ray device.

### BACKGROUND OF INVENTION

Mitral valve insufficiency, often also called “mitral regurgitation”, is a cardiac valve defect that may occur in humans. It involves an inability to close or a “leakiness” of the mitral valve of the heart, which, during the so-called ejection phase (systole), leads to a reverse flow of blood from the left ventricle into the left atrium.

The mitral valve functions as a valve between the left atrium and the left ventricle of the heart. In the filling phase (diastole) of the ventricle, it opens and thus enables the inflow of blood from the left atrium. At the onset of the ejection phase (systole), the suddenly rising pressure in the ventricle leads to closure of the mitral valve and thus to a “sealing off” of the atrium. In this way, the pressure in the atrium is only about 8 mmHg, while, at the same time, in the ventricle the systolic pressure of approximately 120 mmHg drives the blood into the main artery (aorta) in the usual way.

To classify mitral insufficiency by degrees of severity, it is known to differentiate between three or four degrees, for example mild, moderately severe and severe mitral insufficiency. In the case of mild mitral insufficiency, the physiological processes are only slightly affected and neither the extent of the leakiness or (regurgitation opening) nor the quantity of blood flowing back (regurgitation volume) reach degrees that could lead to an anomaly of the pressure in the left atrium and in the pulmonary veins and the pumping capacity of the heart.

Severe mitral insufficiency, which can, for example, be defined as a regurgitation opening of more than 40 mm² and a regurgitation volume of more than 60 ml, can result in serious and sometimes life-threatening changes. In such cases, there is principally a considerable increase in the pressure in the atrium and hence also in the pulmonary veins, wherein the pressure can be as high as 100 mmHg, which, even when the pulmonary vessels are in a normal condition, can result in immediate pulmonary edema. In addition, the resulting predominant reverse flow of blood can cause inadequate ejection output into the aorta and consequently deficient perfusion of other organs. Such problems occur in the acute stage.

If the acute stage is withstood or if the mitral insufficiency develops over a longer period of time, the result is a series of chronic adaptation processes in the heart and the pulmonary vessels. Persistent pressure and volume loading of the atrium causes the enlargement thereof, wherein the atrium volume can often triple or quadruple in size within months or years. Over the course of time, this dilation also reduces the pressure-increasing effect of the regurgitation volume in pulmonary circulation. In addition, the pressure loading also causes an enlargement of the left ventricle, which now, with every heart beat, in addition to the actual quantity of blood required, also has to pump the regurgitation volume. Although, by means of the so-called Frank-Starling mechanism, this dilation is able, on the one hand, to increase the stroke volume, it leads, on the other hand, to a “vicious circle”, if on the expansion of the ventricle, the geometry of the mitral valve is also disturbed and in this way its insufficiency is increased still further.

In order to avoid serious and high-risk interventions as far as possible in the case of mitral valve insufficiency, researchers have turned to the field of minimally invasive cardiac valve interventions. For this, it has been suggested that a clip be used to in order to clamp together the leaflets of the mitral valve in a minimally invasive way. A clip of this kind is known under the name MitraClip and is described, for example, in more detail in US 2005/0149014 A1 and US 2006/0184203 A1. The procedure for using a clip device of this kind is described, for example, in the article “Step-by-Step Guide for Percutaneous Mitral Leaflet Repair” by Mehmet Cilingiroglu et al., Cardiac Interventions Today, edition dated July/August 2010, pages 69-76. According to this, the aforementioned MitraClip is used to clamp the leaflets of the mitral valve in a minimally invasive intervention. This results in better closure of the mitral valve and prevents or reduces the reverse flow of blood from the left ventricle to the left atrium.

The procedure described in the cited article uses an X-ray device with a C-arm to record fluoroscopic images and an ultrasound device; the MitraClip is brought to the site to be treated by a guide catheter. The workflow described therein entails the introduction of the guide catheter into the right atrium using fluoroscopy, following which the septum (the partition wall) of the heart is punctured using fluoroscopy and ultrasound imaging in order to move the guide catheter from the right atrium into the left atrium. Again using fluoroscopy and ultrasound imaging, the MitraClip is positioned above or on the leaflets of the mitral valve. Finally, the MitraClip is clamped on the leaflets of the mitral valve, following which the tightness of the closed mitral valve and the permeability of the open mitral valve are checked using Doppler ultrasound imaging, wherein repositioning may be necessary. If the results are satisfactory, the MitraClip is finally secured and the catheter can be removed again under fluoroscopic monitoring, wherein it is possible to check in a final fluoroscopic image whether all the catheters and tools have been completely removed.

One drawback of these devices and the associated workflow is the fact that fluoroscopy and ultrasound imaging (frequently echocardiography through the esophagus, “transesophageal echocardiography—TEE”) are not able to depict the anatomy of the right and left atria sufficiently well. Therefore, it is, on the one hand, difficult to identify a suitable puncture site in the septum and, on the other, it is difficult to check how far the catheter reaches into the left atrium so that there is a risk of damage to the left wall of the atrium. If the puncture site is poorly chosen, for example too high or too low, there is a risk that, due to the restricted mobility of the catheter, the MitraClip cannot be correctly positioned or correctly secured on the mitral leaflets.

The aforementioned article by Mehmet Cilingiroglu et al. refers to the importance of optimum positioning of the transseptal puncture with respect to the outcome of the procedure: “Because of the need to approach the mitral valve at appropriate angles in all three planes of the valve to ensure successful and adequate grasping of the mitral leaflets, it is critical that the transseptal puncture is placed relatively posterior and relatively high in the fossa ovalis. This high puncture allows an adequate working space and distance above the mitral leaflets (ideal height from the annulus should be 3.5-4 cm) for the delivery catheter manipulations, clip opening, and clip retraction during the grasping.”

The later-published US patent application with the U.S. application Ser. No. 12/881,925 of the applicant, the disclosure content of which is fully incorporated by reference into the disclosure content of the present invention, describes a device and a method for minimally invasive therapy of mitral regurgitation and proposes that it is no longer mandatory to use an ultrasound device, but instead an X-ray device with a C-arm can be used in order to take both CT-like and fluoroscopic images, wherein a CT-like image is superimposed on a fluoroscopic image during the use of the catheter. Here, it can be provided that segmentation is performed and is also visible in the superimposed representation. The superimposed data is used to assist the guidance of the catheter. Therefore, although it is suggested that features are extracted from the three-dimensional CT-like image data record by segmentation which can then be superimposed on fluoroscopic images, even this does not disclose a specific, advantageous variant for finding a suitable puncture site.

### SUMMARY OF INVENTION

The invention is therefore based on the object of disclosing an improved method for assisting in the identification and use of a suitable puncture site.

To achieve this object, according to the invention, the following steps are provided with a method of a type named in the introduction:
the recording of a three-dimensional image data record showing at least one anatomical structure in the region of the septum, in particular in the heart,
the determination of at least one item of septum information showing the position of the septum in the image data record and/or at least one item of additional information derived from the position of at least one anatomical structure, influencing or showing the selection of the puncture site,
during the intervention, the continuous recording of fluoroscopic images of the region and displaying of the current fluoroscopic image superimposed with the septum information and/or the additional information and/or information derived from the septum information and/or the additional information based on a registration of the image data record with the fluoro scopic images.

It is, therefore, proposed that a three-dimensional image data record in particular of the heart be used to determine essential information to enable the correct puncture of the septum. It is possible, on the one hand, to determine, either manually or preferably automatically, septum information that describes the position of the septum in three-dimensional space at least approximately. This determination of the position of the septum as an anatomical structure can now be further supplemented by additional information, which influences or even shows the selection of the puncture site and which is derived from the position of at least one anatomical structure. However, it is also conceivable that the additional information is determined and superimposed regardless of the position of the septum, that is also without the determination of septum information, wherein then the person performing the intervention can himself identify the position of the septum from the image. Therefore, a manual or preferably automatic evaluation of the three-dimensional image data record is performed which results in the additional information, which is, for example, an auxiliary line or another auxiliary superimposition, which assists in the selection of the puncture site and the guidance thereto. In particular, a desired puncture site can itself be specified as additional information, in particular during planning, at least partially automatically. Suitable superimposition of the septum information and the additional information into the current fluoroscopic images, from which the position of the catheter is evident, provides the person performing the intervention with an excellent aid that improves the guidance of the catheter in that the difficult task of selecting and finding the puncture site can be made much easier for him.

It is therefore possible according to the present invention to plan the puncture site on a three-dimensional representation of the three-dimensional image data record, that is the relevant heart structure, and to visualize this planning optimally to the person performing the intervention on the fluoroscopic image as guidance. This enables a better selection of a puncture site and more precise guidance of the catheter to the puncture site.

The method according to the invention can be used particularly advantageously for the treatment of mitral valve insufficiency. As described in the introduction, in this case it is, for example, conceivable, to clamp together the leaflets of the mitral valve, in particular by means of a MitraClip. To do this, it is necessary, to go from the right atrium through the septum into the left atrium of the heart. In this context, it can with particular advantage be provided that, when treating mitral valve insufficiency, in particular when clamping together the leaflets of the mitral valve, as an intervention, at least one item of mitral valve information is determined describing the position of the mitral valve and at least one item of additional information is derived therefrom. In the case of an intervention on the mitral valve, the relevant anatomical structure is, therefore, the mitral valve itself, wherein, in the example of the application of a MitraClip to the leaflets of the mitral valve, it is necessary to navigate very precisely with the catheter, wherein this has to be introduced into the left atrium and then can, for example, be bent so that the MitraClip ideally lies precisely in the region of the closing mitral leaflets. Therefore, as already explained in the representation of the prior art, the distance of the puncture site from the mitral valve, in particular the height above the mitral valve, can play an important role.

In order to facilitate improved planning and guidance, it can be provided that the mitral valve information is determined as the course of the annulus of the mitral valve and from this additional information is determined, allowing for a predefined distance and/or distance range, as a one- or two-dimensional orientation range of the septum and/or the wall of the left atrium and/or an orientation plane intersecting the septum and/or the wall of the left atrium. Therefore, when the position of the mitral valve has been determined as an anatomical structure, it is proposed that this be described by the course of the annulus, that is of the edge, of the mitral valve. This is used as a basis in order to determine, as additional information, for example, an orientation range, in which the puncture site should ideally lie, or an orientation plane, the line of intersection whereof with the septum (as a one-dimensional orientation range) offers orientation when identifying a suitable puncture site. If this additional information is determined at an early stage, it is possible, for example by manual interaction in the planning phase, in particular in a three-dimensional representation of the anatomy, on the basis of the three-dimensional image data record, to determine manually or also automatically an optimal puncture site as further additional information.

In a concrete embodiment of the present invention, it is suggested that a mitral valve plane derived from the course of the annulus is determined, wherein the orientation range and/or the orientation plane are at the predetermined distance or distance range from the mitral valve plane. A plane of this kind can be determined from the annulus of the mitral valve, which usually has an approximately saddle-like shape, for example as an equalization plane using methods that are known in principle, but other possibilities are also conceivable, for example the selection of at least three points through which the plane should extend or the like. It is then particularly simple to use the predetermined distance or distance range. When positioning a MitraClip, a preferable predetermined distance or distance range is in particular a distance from three to five centimeters, for example 3.5-4 cm, as suggested in the article by Mehmet Cilingiroglu et al. cited in the introduction. However, it is also noted at this point that the optimal puncture site does not necessarily have to lie in the center of the orientation range inside the septum or in the center of the line of intersection of the orientation planes with the septum.

In a particularly advantageous embodiment of the present invention, it can also be provided that further intervention information relevant for the intervention is determined in the image data record and shown superimposed on the current fluoroscopic image. Therefore, further concrete embodiments are conceivable to simplify the planning and the navigation for the person performing the intervention and permit a more exact intervention. For example, it can be provided that risk information showing a position of regions at risk from the catheter, in particular the auricle and/or the pulmonary veins, is used as intervention information. In particular in the example of the positioning of a MitraClip, here reference is made to the auricle and the pulmonary veins, which are at risk if the catheter is inserted too far into in the left atrium. Therefore, such regions can be particularly advantageously shown highlighted in the fluoroscopic image in order to avoid problems here at an early stage. It is further advantageous for navigation information showing the position of a navigation target, in particular a valve line to which the leaflets of the mitral valve are connected, and/or the surrounding anatomy, to be determined. In an intervention to rectify a mitral valve insufficiency, it is particularly advantageous to extract navigation information describing the valve line (coaptation line), wherein this determines, for example, how a catheter bearing a MitraClip should be correctly oriented. Therefore, additional information of this kind also enables an important display providing improved assistance to the person performing the intervention. However, further anatomical structures in the environment could also serve as navigation aids and hence corresponding navigation information can also be determined in this regard.

To determine the position of at least one anatomical structure in the three-dimensional image data record, advantageously segmentation and/or the use of an anatomical atlas can be provided, wherein particularly advantageously the determination is performed at least partially automatically. Basic procedures for the automatic and/or manual segmentation of anatomical structures are known. In particular in cases where anatomical structures cannot be determined with sufficient clarity from the image data record, it can be advantageous to refer to an anatomical atlas in order to be able to at least approximate their position. If, for example, an intra-operative image data record recorded with an X-ray device with a C-arm is used as a three-dimensional image data record, frequently the septum cannot be clearly identified therein so that here it may be advantageous to refer to an anatomical atlas and then to approximate the septum, for example with a simple shape such as a circle or an ellipse. This is also conceivable with other anatomical features, for example the annulus of the mitral valve, which can be approximated as a circle, or the like. Concrete algorithms for segmentation in three-dimensional image data records do not need to be explained in more detail here since they are already known to the person skilled in the art from the prior art.

A preoperative magnetic resonance image data record and/or a computed tomography image data record and/or an ultrasound image data record can be used as a three-dimensional image data record, wherein in principle obviously also a plurality of three-dimensional image data records can be used. In this case, preoperative image data records generally have the advantage that they are selected such that the relevant anatomical structures can be clearly identified and therefore simple, in particular fully automatic, segmentation is enabled. However, in this case another registration with the fluoroscopic images is necessary so that it can be provided that, when using a preoperative image data record, registration is performed with the fluoroscopic images by 3D-2D registration or by 3D-3D registration with a three-dimensional registration data record recorded by means of the X-ray device used to record the fluoroscopic images.

Methods for 3D-2D registration and for 3D-3D registration are already known in principle from the prior art. If a registration data record, which can also be used as an image data record, is to be recorded, the X-ray device used to record the fluoroscopic images can advantageously be an X-ray device with a C-arm so that a CT-like (computed-tomography-like) registration data record can be generated in that projection images are recorded from different projection directions and reconstructed to form a three-dimensional registration data record. A registration data record of this kind to be recorded on a patient who is already in position directly before the intervention is advantageously generated at a low dose.

However, additionally or alternatively it is also conceivable for the three-dimensional image data record to be recorded at the site of intervention by means of a X-ray device comprising a C-arm, which is also used to record the fluoroscopic images. In this case, only one single image recording device, namely the X-ray device, is necessary, wherein a rotation of the C-arm enables the recording of projection images from different directions from which a CT-like three-dimensional image data record can be reconstructed. Since the fluoroscopic images are also recorded with the X-ray device and the three-dimensional image data is recorded with a patient who is already in position, that is intraoperatively, no further registration is required. The data are registered inherently with each other.

In a particularly expedient development of the method according to the invention it can further be provided that, when using a X-ray device adjustable for different projection directions, in particular comprising a C-arm, to record the fluoroscopic images, at least one projection direction to record the fluoroscopic images is determined and adjusted automatically as a function of the septum information and/or at least one item of additional information and/or determined manually with reference to a representation of the three-dimensional data record and/or the septum information and/or the additional information. It is therefore advantageously conceivable to select a suitable C-arm angulation based on the extracted structures and additional information so that the fluoroscopic images represent an ideally suitable viewing direction for the current phase of the intervention. In this case, the determination does not mandatorily have to take place automatically; it is also possible that for this to take place manually with reference to a representation of the three-dimensional image data record or a representation derived therefrom. Then, for example, the representation can be turned in a specific direction, which the person performing the intervention wishes to see. A projection direction, and hence an angulation, of the C-arm, is then automatically determined and actuated.

In this case, it is advantageous if, to assist the puncture of the septum, a first projection direction standing perpendicularly to the septum and/or parallel to a plane dependent upon the position of the mitral valve, in particular parallel to the mitral valve plane and/or the orientation plane, is selected. In a projection direction of this kind, ideally standing perpendicularly to the septum, it can be checked in an excellent way whether a planned or currently desired puncture site has actually been correctly reached. Since the necessary backgrounds are known anyway from the septum information and the additional information, this adjustment can particularly advantageously take place automatically as can the preceding determination of the optimal projection direction.

In this context, it is further expedient for at least one fluoroscopic image from another second projection direction, in particular standing perpendicularly to the first projection direction, to be recorded and displayed. This is particularly useful if it is advisable to check from another direction, if, therefore, it is to be determined, for example, whether the catheter has already moved from the right atrium into in the left atrium or how far it has advanced, in particular also with respect to risk information, as already described above. Such control recording is preferably performed parallel to the septum so that a puncture of the septum is easily identified.

In this context, reference is made to the fact that with the method according to the invention, in particular with respect to the exemplary embodiments showing the angulation of the C-arm of an X-ray device derived from the information, it can be advantageous to use a biplane-X-ray device since then it can, for example, be provided that fluoroscopic images can be recorded perpendicularly to the septum and parallel to the septum simultaneously hence achieving optimal orientation for the person performing the intervention.

In addition to the method, the invention also relates to an X-ray device comprising a control device designed to carry out the method according to the invention. This is particularly advantageously an X-ray device with a C-arm opposite to which an X-ray tube and X-ray detector are arranged. The C-arm can be swiveled around a patient positioned on a patient bed so that projection images can be recorded from different projection directions from which the three-dimensional image data record can then be reconstructed. In addition, different angulations of the C-arm are possible from which the fluoroscopic images can then be recorded. The control device is particularly advantageously embodied to determine the septum information, the additional information and optionally further information from the three-dimensional image data record at least partially or even wholly automatically and to display it at least partially additionally to the fluoroscopic images and superimposed thereon, for which a corresponding display and operating device is provided. All embodiments relating to the method according to the invention can be similarly transferred to the X-ray device according to the invention so that these are also able to achieve the advantages of the present invention.

### BRIEF DESCRIPTION OF THE DRAWINGS

Further advantages and details of the present invention will emerge from the exemplary embodiments described below and with reference to the drawing, which shows:

FIG. 1 an X-ray device according to the invention,

FIG. 2 a schematic sketch of the left atrium of a heart,

FIG. 3 a workflow plan of the method according to the invention,

FIG. 4 a schematic sketch of the left atrium with auxiliary constructs used to determine the additional information,

FIG. 5 a schematic representation of the left atrium with segmented anatomical structures, the septum information and the additional information,

FIG. 6 a schematic sketch of information displayed from a first projection direction and

FIG. 7 a schematic sketch of information displayed from a further projection direction.

### DETAILED DESCRIPTION OF INVENTION

The present invention will now be discussed using the example of a minimally invasive intervention in which the leaflets of the mitral valve are to be clamped together by means of a so-called MitraClip in order to treat mitral valve insufficiency. Here, a guide catheter is used, which is initially introduced into the right atrium of the heart where the septum is punctured and the catheter is then guided into the left atrium from where the actual treatment is performed. Here, it is important that the puncture of the septum takes place at a suitable puncture site, for which the method according to the invention provides a plurality of assistance options, wherein however, assistance options extending beyond the object of the present invention are also provided during the performance of the minimally invasive intervention.

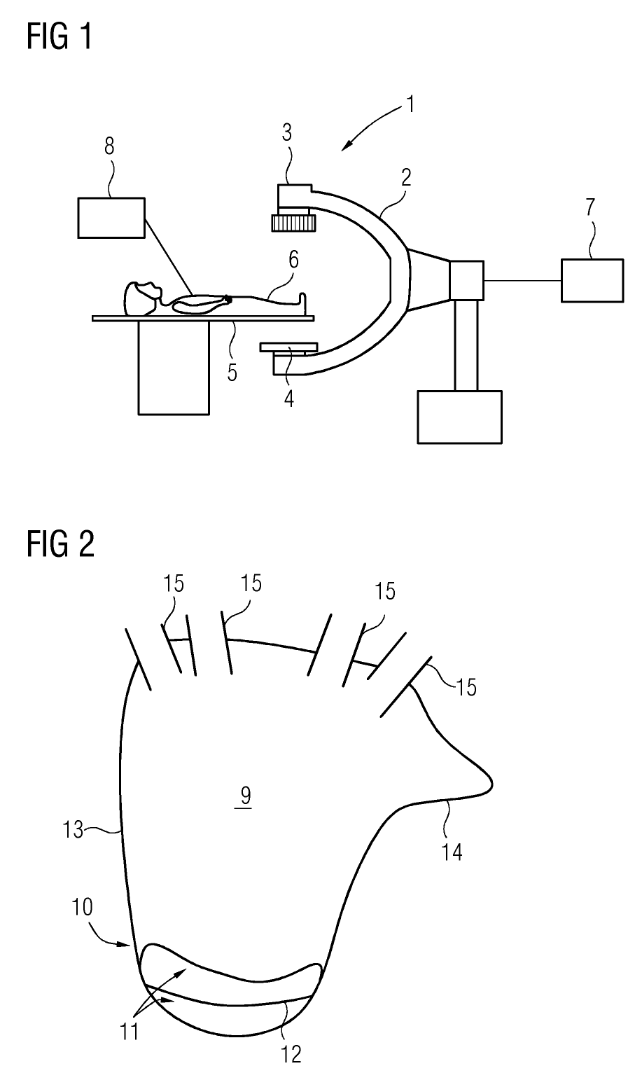

FIG. 1 shows an X-ray device 1 according to the invention. This comprises a C-arm 2 opposite which an X-ray tube 3 and an X-ray detector 4 are arranged. The C-arm 2 can be swiveled about a patient bed 5 on which a patient 6 can be positioned. It is therefore possible to use the X-ray device 1 to record a three-dimensional, CT-like image data record by recording a plurality of projection images from different projection directions so that it is not mandatory to use preoperative three-dimensional image data records. In addition, the X-ray device 1 is designed to record fluoroscopic images at different projection directions, that is different angulations of the C-arm 2.

FIG. 1 also shows a schematic representation of a guide catheter 8 with which the MitraClip is to be brought to its location of use. The guide catheter 8 can be clearly seen in fluoroscopic images taken by the X-ray device 1.

The operation of the X-ray device 1 is controlled by a control device 7, which not only the controls the image recording but is simultaneously designed to evaluate image data and to generate suitable representations to be displayed, specifically to carry out the method according to the invention, which will be described in more detail below.

FIG. 2 shows a schematic sketch of the anatomy of the left atrium 9 of a heart. The left atrium 9 is separated from the left ventricle of the heart, which is not shown in more detail here, by the two leaflets 11 of the mitral valve 10. The valve line 12 (coaptation line) shows where the leaflets 11 touch when the mitral valve 10 is closed.

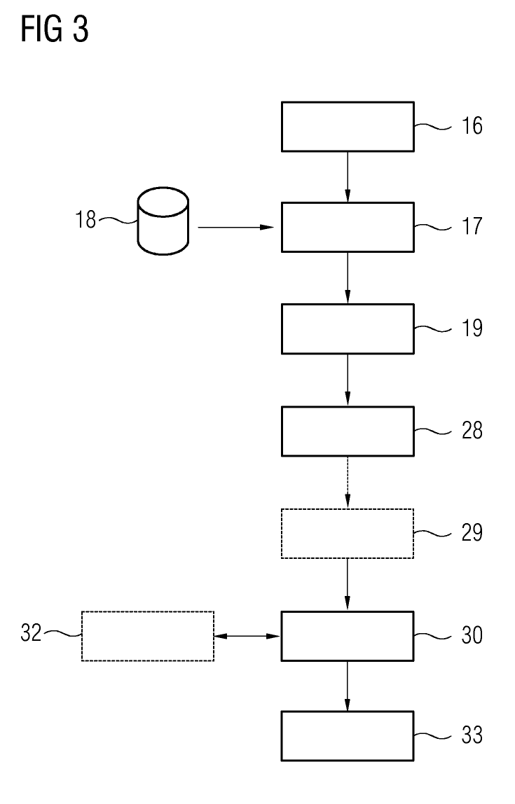

FIG. 2 also shows the septum 13, that is the partition wall with the right atrium, which is also not shown in any more detail for reasons of clarity. Further identifiable anatomical structures are the auricle 14 and pulmonary veins 15. These anatomical structures are relevant for the optimal performance of the minimally invasive intervention and so they should also be taken into account in the method according to the invention, which will now be explained in more detail with respect to FIG. 3.

Initially, in a step 16, a three-dimensional image data record of the heart, in particular of the left atrium, is recorded. In this case, there are substantially two possibilities. On the one hand, it is conceivable to record the three-dimensional image data record preoperatively, for example as a magnetic resonance image data record, a computed tomography image data record or an ultrasound image data record. Alternatively or additionally, it is preferred according to the invention to use the current information to record an intraoperative three-dimensional image data record with a patient 6 who is already in position by means of the X-ray device 1.

In a step 17, the annotation and the planning of the minimally invasive intervention are performed. For this, initially, anatomical structures are segmented, preferably automatically by the control device 7, in such a way that their position is known and further information can be derived therefrom. In addition to the actual three-dimensional image data record, here account is taken of data from an anatomical atlas 18, for example in order to be able to identify the septum at least approximately if the septum 13 cannot be clearly identified on the three-dimensional image data record recorded by the X-ray device 1.

In this case, the position of the following relevant anatomical structures is determined by segmentation or approximation: left atrium 9, mitral valve 10, septum 13, auricle 14 and pulmonary veins 15, wherein obviously other further anatomical structures can also be considered. Septum information indicates the position of the septum 13. Corresponding further information is available with respect to the wall of the left atrium 9, the mitral valve 10, the auricle 14 and the pulmonary veins 15.

In a step 19, this position information, which is already available, is now further processed in order to determine additional information with respect to the septum puncture and intervention information, concrete risk information and navigation information.

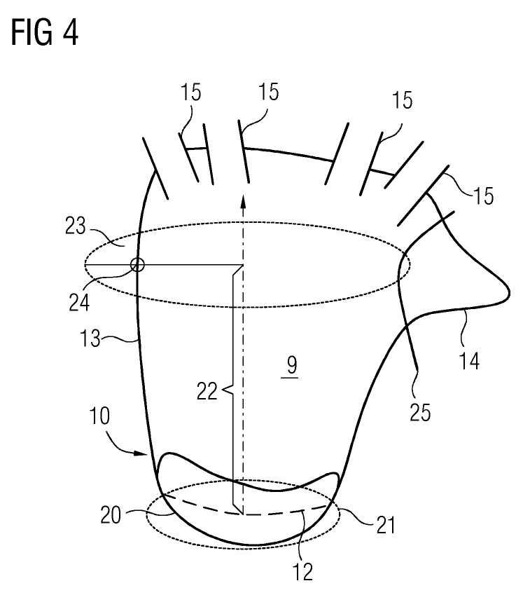

This is explained in more detail by the schematic representation in FIG. 4. In order to obtain important information for the puncture, initially mitral valve information is determined. In the present case, initially the annulus 20, that is the edge, of the mitral valve 10 is determined—this usually has a substantially saddle-like shape. Information on the course of the annulus 20 is now used in order to determine a mitral valve plane 21, for example, in that the equalization plane is determined from the course of the annulus 20 or predetermined points, in particular at least three predetermined points, for forming a plane are considered.

Now, it is known that it is advantageous to introduce the catheter 8 into the left atrium 9 at a certain distance or distance range above the mitral valve plane 21. In the example shown in FIG. 4, this information is present as a predetermined distance 22 measuring 3.554 cm. It is also possible to select a distance range, but this is not shown here for purposes of clarity. Initially, now, an orientation plane 23 lying at a height with the predetermined distance 22 is determined from the mitral valve plane 21. Hence, points of intersection 24 of the orientation plane 23 with the wall of the left atrium 9 and in particular the septum 13 (known from the septum information) represent a good orientation aid to where the puncture of the septum 13 should ideally be performed, here in the form of a line, that is a one-dimensional orientation range. In the present case, this one-dimensional orientation range is considered further as additional information.

It is noted at this point that additional information, which in the present case is determined completely automatically by the control device 7 can also, in particular in conjunction with the three-dimensional image data record or a representation derived therefrom, be displayed to the person performing the intervention for purposes of further planning, who can then, for example, also manually select as further additional information a desired puncture site as one of the points of intersection 24. However, an automatic determination of a puncture site by the control device 7 is also conceivable.

In the present case, further useful intervention information for assisting the person performing the intervention is derived, wherein, in the present case, the position of the actual pulmonary vein 15 represents risk information, exactly like the boundary 25 of the auricle 14, since these anatomical structures should not be damaged by the catheter 8 during the intervention, that is they indicate regions that are better avoided. As further navigation information (as part of the intervention information), in the present case, the valve line 12 is automatically determined by the control device 7, which represents important information with respect to the orientation of the catheter 8 and therefore of the MitraClip.

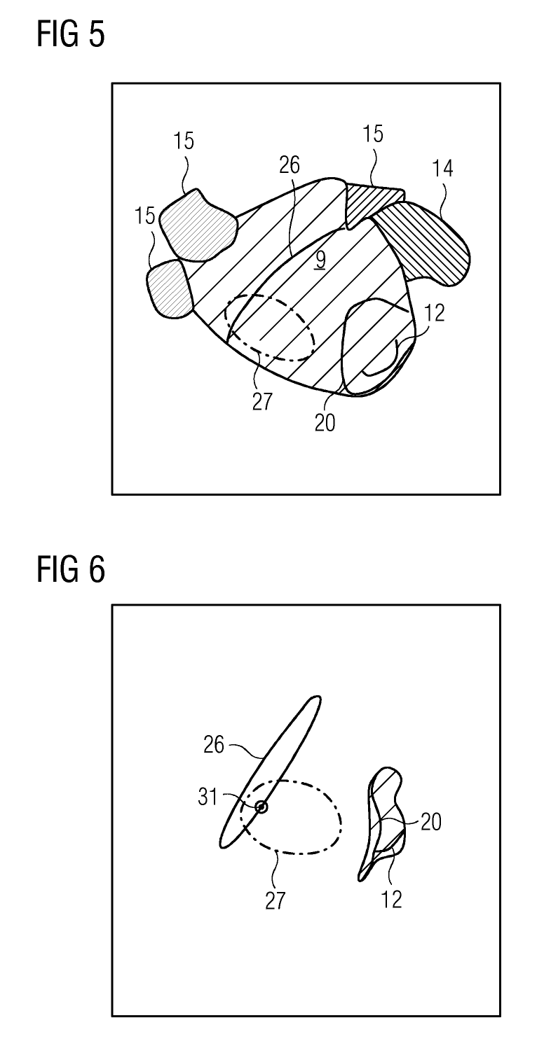

FIG. 5 is a schematic sketch of a possible representation of the final information obtained, which advantageously should also be available during the actual intervention with the catheter 8. Initially displayed are the segmented anatomical structures, that is the left atrium 9, and the structures auricle 14 and pulmonary veins 15 to be displayed as risk information. The annulus 20 of the mitral valve 10 and the valve line 12 (coaptation line) can also be identified. The septum 13 is indicated by a boundary line 27, which has an area of intersection with the one-dimensional orientation range 26 indicating the ideal place for a puncture of the septum 13. In the present example, therefore, the boundary line 27 represents the septum information, the annulus 20 and the orientation range 26 represent additional information and the valve line 12 and the segmented anatomical structures 9, 14 and 15 represent further intervention information.

In a step 28, the planning is then completed and the actual intervention with the catheter 8 starts. Then, in an optional step 29, if a preoperative three-dimensional image data record was used, a registration of the preoperative three-dimensional image data record (and therefore of the information determined) with the fluoroscopic images to be recorded is achieved, for example by a known 3D-2D registration algorithm or by a 3D-3D registration algorithm in conjunction with a three-dimensional registration data record recorded by means of the X-ray device 1. If a three-dimensional image data record of the X-ray device 1 recorded anyway on a patient 6 already in position is now used as the three-dimensional image data record, no further registration is required since the same X-ray device 1 is used.

While the intervention is now being performed, which means the catheter 8 is brought to the mitral valve 10, continuous monitoring is performed by recording fluoroscopic images and displaying these on a display device of the X-ray device 1, for example a monitor. This takes place in step 30. Here, initially the catheter 8 is introduced into the right atrium of the heart under fluoroscopic monitoring. From this time at the latest, at least a part of the information determined is displayed superimposed on the current fluoroscopic image as is explained in more detail for example by FIG. 6. For purposes of simplicity, the fluoroscopy data is not shown here, only the additionally displayed information, in this phase of the intervention, the boundary line 27 of the septum 13, the orientation range 26, in the present case with a marked desired puncture site 31, and the annulus 20 of the mitral valve 10, the valve line 12 and, here only indicated, the leaflets 11 of the mitral valve 10. In the present case, the angulation of the C-arm 2 was selected such that the projection direction stands perpendicularly to the septum 13 so that there is an ideal view of whether the tip of the catheter 8 (not shown here for purposes of simplicity) identifiable in the fluoroscopic image will hit the desired puncture site 31. The necessary angulation angle of the C-arm 2 was determined and adjusted automatically by the control device 7, step 32. This is possible once the position of the septum 13 with the boundary line 27 is known and therefore the corresponding projection direction, that is also the angulation angle, can be determined automatically.

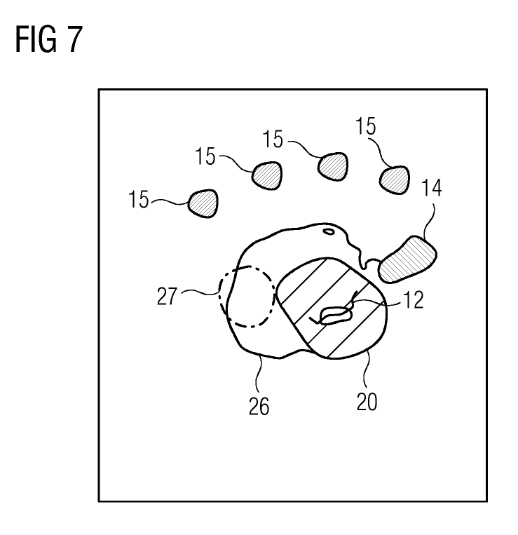

It is advantageous if, at the latest after the puncture of the septum 13 and the penetration of the catheter 8 into the left atrium 9, another setting is selected, for example a setting running substantially parallel to the septum 13, as indicated by FIG. 7. This shows how far the catheter 8 has already penetrated the left atrium 9. The additional representation of the risk information—that is the position of the auricle 14 and the pulmonary veins 15—enables the person performing the intervention to navigate well and reliably in order finally—optionally with a further manual or automatic change to the projection angle—to bring the intervention to an end. This is performed substantially as described in the introduction, which means that the MitraClip is attached in such a way that the leaflets 11 of the mitral valve 10 are clamped together, wherein, to set the right orientation, navigation with reference to the valve line 12 was possible at an early stage. After the removal of the guide catheter and optionally further tools used from the body of the patient, the intervention is then ended in step 33 in exactly the same way as the recording of fluoroscopic images.

It should also be noted at this point that it also possible within the scope of the present invention to work without septum information so that only the additional information is determined and superimposed on the image. For example, here, a plane or a circular area of a specific size can be determined above the mitral valve and superimposed on the fluoroscopic images without concrete details of the location of the septum.

1. A method for assisting a minimally invasive intervention with a catheter involving a puncture of a septum, comprising:
recording a three-dimensional image data record showing an anatomical structure in a region of the septum;
determining a septum information showing a position of the septum in the image data record and additional information derived from the position of the septum influencing a selection of a puncture site; and
continuously recording fluoroscopic images of the region by an X-ray device and displaying a current fluoroscopic image during the intervention,
wherein the current fluoroscopic image is superimposed with the septum information and/or the additional information.

2. The method as claimed in claim 1, wherein the intervention comprises insufficiency of a mitral valve, and wherein information of the mitral valve describing a position of the mitral valve is determined and additional information is derived therefrom.

3. The method as claimed in claim 2, wherein the insufficiency of the mitral valve comprises clamping leaflets of the mitral valve.

4. The method as claimed in claim 2, wherein the information of the mitral valve is determined as a course of annulus of the mitral valve, and wherein the additional information is determined allowing a predefined distance and/or a predefined distance range as an orientation range of the septum, and/or as an orientation range of a wall of a left atrium, and/or as an orientation range of an orientation plane intersecting the septum and/or the wall of the left atrium.

5. The method as claimed in claim 4, wherein a mitral valve plane is derived from the course of the annulus, and wherein the orientation range and/or the orientation plane comprise the predetermined distance or the predetermined distance range to the mitral valve plane.

6. The method as claimed in claim 1, wherein further intervention information relevant for the intervention is determined in the image data record and is superimposed on the current fluoroscopic image.

7. The method as claimed in claim 6, wherein the further intervention information comprises a risk information showing a position of an area at risk from the catheter, and/or navigation information showing a position of a navigation target, and/or a surrounding anatomy.

8. The method as claimed in claim 7, wherein the area comprises an auricle and/or pulmonary veins, and wherein the navigation information comprises a valve line to which leaflets of a mitral valve are connected.

9. The method as claimed in claim 1, wherein the position of the anatomical structure is determined at least partially automatically by a segmentation and/or using an anatomical atlas.

10. The method as claimed in claim 1, wherein the three-dimensional image data record comprises a preoperative magnetic resonance image data record and/or a preoperative computed tomography image data record and/or a preoperative ultrasound image data record.

11. The method as claimed in claim 10, wherein the preoperative image data record is registered with the fluoroscopic images.

12. The method as claimed in claim 1, wherein the three-dimensional image data record is recorded at the intervention by the X-ray device from different projection directions.

13. The method as claimed in claim 12, wherein at least one projection direction is determined and adjusted automatically as a function of the septum information and/or the additional information.

14. The method as claimed in claim 13, wherein a first projection direction perpendicular to the septum and/or parallel to a plane dependent upon a position of a mitral valve is selected.

15. The method as claimed in claim 14, wherein the plane is parallel to a mitral valve plane and/or an orientation plane.

16. The method as claimed in claim 14, wherein a fluoroscopic image from a second projection direction perpendicular to the first projection direction is recorded and displayed.

17. The method as claimed in claim 1, wherein the septum is a heart.

18. An X-ray device for assisting a minimally invasive intervention with a catheter involving a puncture of a septum, comprising:
an X-ray tube;
an X-ray detector for continuously recording fluoroscopic images of an anatomical structure in a region of the septum during the intervention; and
a control device adapted to:
determine a septum information showing a position of the septum in the fluoroscopic images and additional information derived from the position of the septum influencing a selection of a puncture site,
superimpose a current fluoroscopic image with the septum information and/or the additional information, and
display the superimposed current fluoroscopic image.
